(19) United States
(12) Patent Application Publication
(10) Pub. No.: US 2004/0185085 A1
(43) Pub. Date: Sep. 23, 2004

(54) Base material for tissue regeneration, implant material, and method of producing implant material

(75) Inventors: Mitsuo Ochi, Hiroshima (JP); Yoshito Ikada, Kyoto (JP); Katsura Sugawara, Aichi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: JAPAN TISSUE ENGINEERING CO., LDT., Gamagori-shi (JP)

(21) Appl. No.: 10/766,876

(22) Filed: Jan. 30, 2004

Related U.S. Application Data
(63) Continuation of application No. PCT/JP02/07532, filed on Jul. 25, 2002.

(30) Foreign Application Priority Data
Jul. 30, 2001 (JP) 2001-230260

(51) Int. Cl. A61F 2/00

(52) U.S. Cl. 424/426

(57) ABSTRACT

A base material for tissue regeneration (10) includes a collagen sponge (11) formed in a three-dimensional shape and a mesh support member (12) that supports the collagen sponge (11) in an externally accessible state. In the structure of the base material for tissue regeneration (10), the mesh support member (12) is provided to surround the collagen sponge (11). Even when the collagen sponge (11) formed in the three-dimensional shape has a difficulty in keeping its shape, the mesh support (12) effectively functions to keep the three-dimensional shape.

### CROSS-REFERENCE TO RELATED APPLICATION

This is a continuation of Application PCT/JP02/07532, filed Jul. 25, 2002, now abandoned.

### BACKGROUND OF THE INVENTION

1. Field of the Invention

The present invention relates to a base material for tissue regeneration that is used to culture cells and reconstruct tissue in vivo and in vitro, as well as to an implant material utilizing the base material.

2. Description of the Prior Art

A number of techniques have been reported recently to culture cells in vitro and implant resulting cultured tissues in a patient. The cells may not be cultured alone, but in many cases, the cells are seeded and cultured on a carrier (base material for tissue regeneration) used as a scaffold of cell proliferation. The carrier has especially important roles to prepare tissues in a three-dimensional shape having a certain depth or height. The recently developed, attention-drawing technique implants a carrier or base material functioning as a scaffold of tissue regeneration and reproduces the tissues in vivo by taking advantage of the self-healing power. This technique is called regenerative medicine or tissue engineering.

Biocompatible materials and bioabsorbable materials are applied to the carrier or the base material for tissue regeneration. Available examples include collagen, hyaluronic acid, polyrotaxane, gelatin, fibronectin, heparin, chitin, chitosan, laminin, calcium alginate, and polyrotaxane hydrogel.

These prior art base materials for tissue regeneration have relatively low strength and difficulties in sufficiently maintaining their long, bulky, or tall shapes. The insufficient strength of the base material for tissue regeneration leads to the difficult handling in the process of culture and implantation and the poor operation ability and imposes the heavy load on the operators.

In order to solve the problems of the prior art materials, the present invention aims to provide an easily handling base material for tissue regeneration and a corresponding implant material. The object of the invention is also to provide a method of producing such an implant material.

### SUMMARY OF THE INVENTION

A first base material for tissue regeneration of the invention includes: a porous carrier that is formed in a three-dimensional shape; and a support member that is provided to surround said porous carrier and supports said porous carrier in an externally accessible state.

In the structure of this base material for tissue regeneration, the support member is provided to surround the porous carrier and thereby effectively maintains the three-dimensional shape of the porous carrier, even if the porous carrier alone has difficulty in maintaining its three-dimensional shape. This facilitates handling of the base material for tissue regeneration. The support member supports the porous carrier in an externally accessible state. When the base material for tissue regeneration is implanted, this arrangement enables the surrounding living tissue to gain access to the porous carrier via the support member and thereby enhances the biocompatibility of the base material. The base material of this invention is thus suitable for tissue regeneration.

The base material for tissue regeneration may be implanted in the living body directly, or may otherwise be processed to make cells held on its porous carrier and then implanted as an implant material in the living body. In the former case, the cells of the living tissue surrounding the implantation site enter the porous carrier via the support member to be proliferated therein and make the implant take. In the latter case, the procedure specifies the type of cells to be held on the porous carrier, corresponding to the living tissue of the implantation site and makes the specified cells held on the porous carrier. The cells held on the porous carrier gain access to the living tissue surrounding the implantation site via the support member. This allows for take of the implant material. Here the cells may be held on part of the porous carrier or on the whole porous carrier. Only one type of cells may be held on the porous carrier, or multiple different types of cells may be held simultaneously on the porous carrier. The porous carrier with the cells held thereon may be subjected to culture.

In the base material for tissue regeneration of the invention, the porous carrier represents a carrier having a large number of pores and has a sponge, honeycomb, or any other equivalent structure, although the sponge structure is preferable. The pore diameter is not specifically restricted, as long as the pore has a size of holding a cell therein. The porous carrier is formed in a three-dimensional shape. The three-dimensional shape may be any of a column, a polygonal column, a cone, a polygonal pyramid, a truncated cone, a truncated polygonal pyramid, and a sphere, or the specific shape of a living region, such as an auditory capsule.

In the base material for tissue regeneration, the support member may not completely surround the whole porous carrier but may cover only part of the porous carrier, as long as the support member functions to maintain the three-dimensional shape of the porous carrier. The support member is preferably any of a mesh support member, a palisade support member, and a perforated plate support member. This structure enables the porous carrier to be supported in an externally accessible state.

In the base material for tissue regeneration, it is preferable that at least one of the porous carrier and the support member is composed of either a biocompatible material or a bioabsorbable material. It is especially preferable that both of the porous carrier and the support member are composed of either the biocompatible material or the bioabsorbable material. The living body hardly recognizes an implanted base material of this composition as foreign, so that the base material of this composition is suitable for tissue regeneration. The bioabsorbable material is decomposed and absorbed after implantation and is thus especially suitable for tissue regeneration.

The biocompatible material and the bioabsorbable material are not restricted specifically. The porous carrier is preferably made of one component or a combination of multiple components selected from the group consisting of collagen, collagen derivatives, hyaluronic acid, hyaluronates, chitosan, chitosan derivatives, polyrotaxane, polyrotaxane derivatives, chitin, chitin derivatives, gelatin, fibronectin, heparin, laminin, and calcium alginate, and said support member is made of one component or a combination of multiple components selected from the group consisting of polylactic acid, polyglycolic acid, polycaprolactone, polylactic acid-polyglycolic acid copolymer, polylactic acid-polycaprolactone copolymer, and polyglycolic acid-polycaprolactone copolymer. Metals like titanium, titanium alloys, stainless steels, cobalt-chromium alloys, and cobalt-chromium-molybdenum alloys, ceramics like alumina ceramics, carbon ceramics, zirconia ceramics, silicon carbide ceramics, silicon nitride ceramics, and glass ceramics, and other bioinert materials are also applicable to the material of the support member. Bioactive matrix materials like hydroxyapatite, calcium phosphate, calcium carbonate, and bioglass are further applicable to the material of the support member.

In the base material for tissue regeneration, the support member preferably has one suture thread. The base material is fixed to the living tissue of the implantation site by means of the suture thread. For the tissue regeneration, it is preferable that the suture thread is made of the biocompatible material or the bioabsorbable material discussed above. For the enhanced production efficiency, the suture thread is preferably composed of the same material as that of the support member.

The base material for tissue regeneration may be implanted after incision of the living tissue surrounding an implantation site. In order to reduce invasion against the patient, arthroscopic surgery is desirable. It is thus preferable that the base material is formed in a specific shape available for arthroscopic surgery. For example, the base material is formed in a columnar shape and has a diameter of 2 to 15 mm in cross section.

A second implant material of the invention includes: a cell-holding carrier that is formed in a three-dimensional shape and holds a cell thereon; and a support member that is provided to surround said cell-holding carrier and supports said cell-holding carrier in an externally accessible state.

In the structure of the implant material, the support member is provided to surround the cell-holding carrier and thereby effectively maintains the three-dimensional shape of the cell-holding carrier, even if the cell-holding carrier is impregnated with a cell suspension or a medium and has difficulty in maintaining its three-dimensional shape. This facilitates handling of the implant material. The support member supports the cell-holding carrier in an externally accessible state. When implant material is implanted, this arrangement enables the surrounding living tissue to gain access to the cell-holding carrier via the support member and to supply the nutrients to the cells held on the cell-holding carrier via the support member. This enhances the biocompatibility of the implant material. The implant material of this invention is thus suitable for tissue regeneration.

In the second implant material of the invention, the cell-holding carrier may be a porous carrier in a three-dimensional shape (for example, carrier having a large number of pores), a gel carrier embedding cells therein, or any other equivalent carrier. The porous carrier may have a sponge, honeycomb, or any other equivalent structure, although the sponge structure is preferable. The pore diameter is not specifically restricted, as long as the pore has a size of holding a cell therein.

The second implant material of the invention may have an artificial graft in a three-dimensional shape that is arranged adjacent to the cell-holding carrier. The artificial graft represents a cell-free artificial material and is not specifically restricted. For example, one of bioinert materials including metals like titanium and ceramics like alumina ceramics, bioactive matrix materials like hydroxyapatite, calcium phosphate, and calcium carbonate, and other materials applicable to implants like artificial bones and artificial joints is adequately selected and used according to the desired shape and strength. In one desirable structure, the support member surrounds the artificial graft as well as the cell-holding carrier, preferably in the externally accessible state. The artificial graft and the support member may be formed integrally of an identical material.

In the second implant material of the invention, it is preferable that the cell includes at least one of epidermal cell, epithelial cell, keratinocyte, fibroblast, chondrocyte, osteoblast, osteocyte, muscle cell, hepatocyte, myocardial cell, their precursor cells, mesenchymal stem cell, and embryonic stem cell (ES cell). It is especially preferable that the cell includes at least one of chondrocyte, osteoblast, osteocyte, their precursor cells, mesenchymal stem cell, and embryonic stem cell (ES cell). The mesenchymal stem cell and the ES cell are undifferentiated and are expected to be differentiated into cells corresponding to the living tissue of each implantation site after implantation. One preferable procedure differentiates such stem cells in vitro into cells corresponding to the living tissue of each implantation site, prior to the implantation.

In one preferable application of the second implant material of the invention, the cell includes chondrocyte held in one half of the cell-holding carrier and either of osteoblast and osteocyte held in the other half of the cell-holding carrier. The implant material of this structure is suitable for implantation in bone/cartilage defects, since the joint has an upper layer of cartilage tissue and a lower layer of bone tissue. Here the terminology ‘half’ does not strictly mean ‘½’. The area of holding the chondrocytes and the area of holding the osteoblasts or osteocytes may be divided at any ratio, and there may be a little overlap.

When the second implant material of the invention has the artificial graft in a three-dimensional shape that is arranged adjacent to the cell-holding carrier, the artificial graft may be artificial bone, and the cell may be chondrocyte. The implant material of this structure is also suitable for implantation in bone/cartilage defects, since the joint has an upper layer of cartilage tissue and a lower layer of bone tissue.

In the second implant material of the invention, the support member may not completely surround the whole cell-holding carrier but may cover only part of the cell-holding carrier, as long as the support member functions to maintain the three-dimensional shape of the cell-holding carrier. The support member is preferably any of a mesh support member, a palisade support member, and a perforated plate support member. This structure enables the cell-holding carrier to be supported in an externally accessible state. In the implant material of the invention, it is preferable that at least one of the cell-holding carrier and the support member is composed of either a biocompatible material or a bioabsorbable material. It is especially preferable that both of the cell-holding carrier and the support member are composed of either the biocompatible material or the bioabsorbable material. The living body hardly recognizes an implanted base material of this composition as foreign, so that the base material of this composition is suitable for tissue regeneration. The bioabsorbable material is decomposed and absorbed after implantation and is thus especially suitable for tissue regeneration. Available examples of the biocompatible material and the bioabsorbable material are given previously. It is preferable that the support member has at least one suture thread. The implant material is fixed to the living tissue of the implantation site by means of the suture thread. The suture thread is preferably composed of the same material as that of the support member.

The second implant material of the invention may be implanted after incision of the living tissue surrounding an implantation site. In order to reduce invasion against the patient, arthroscopic surgery is desirable. It is thus preferable that the implant material is formed in a specific shape available for arthroscopic surgery. For example, the implant material is formed in a columnar shape and has a diameter of 2 to 15 mm in cross section.

A third implant material production method of the invention produces the second implant material of the invention and adopts either of processes (1) and (2) to obtain said cell-holding carrier: (1) differentiating mesenchymal stem cell into an object cell, preparing a cell suspension of the differentiated cell, and seeding the prepared cell suspension onto a preliminary carrier, which is capable of holding a cell and is formed in a three-dimensional shape, so as to obtain said cell-holding carrier; and (2) seeding a cell suspension containing mesenchymal stem cell onto a preliminary carrier, which is capable of holding a cell and is formed in a three-dimensional shape, and differentiating the mesenchymal stem cell held in said preliminary carrier into an object cell, so as to obtain said cell-holding carrier.

This arrangement desirably relieves invasion against the patient in the implant surgery. One available method of producing the implant material obtains the tissue from the surrounding of an implantation site, cultures the obtained tissue cells, and makes the cultured cells held on the preliminary carrier. This method, however, imposes a significant invasion against the patient and is thus not desirable. For example, treatment of a joint cartilage site requires biopsy of the cartilage tissue from a unloaded healthy site to obtain chondrocytes for culture. In order to reduce invasion against the patient, one preferable procedure obtains the marrow cells from a patient and differentiates the undifferentiated mesenchymal stem cells among the obtained marrow cells into object cells (that is, cells corresponding to the living tissue of an implantation site). The object cells obtained by differentiating the mesenchymal stem cells may be seeded on the preliminary carrier. Alternatively the mesenchymal stem cells may be seeded on the preliminary carrier and be differentiated into object cells on the preliminary carrier. In the latter case, it is expected that the mesenchymal stem cells are differentiated into cells corresponding to the surrounding living tissue after the implantation. The available procedure may thus seed the mesenchymal stem cells onto the preliminary carrier and immediately implant the preliminary carrier in the living body for in-vivo differentiation of the mesenchymal stem cells.

The preliminary carrier may be a porous carrier that is capable of holding cells (for example; the porous carrier included in the first base material for tissue regeneration of the invention), a gel carrier that is capable of embedding cells therein, or any other equivalent carrier.

The cell-holding carrier having chondrocytes in one half and either osteoblasts or osteocytes in the other half is obtained by any of the following methods:

(1) seeding a cell suspension containing mesenchymal stem cells into one half of the preliminary carrier and culturing and differentiating the mesenchymal stem cells on the carrier into chondrocytes and subsequently seeding either osteoblasts or osteocytes obtained by differentiating mesenchymal stem cells into the other half of the preliminary carrier;

(2) seeding a cell suspension containing mesenchymal stem cells into one half of the preliminary carrier and culturing and differentiating the mesenchymal stem cells on the carrier into chondrocytes and subsequently seeding a cell suspension containing mesenchymal stem cells into the other half of the preliminary carrier and culturing and differentiating the mesenchymal stem cells on the carrier into either osteoblasts or osteocytes;

(3) seeding chondrocytes obtained by differentiating mesenchymal stem cells into one half of the preliminary carrier and subsequently seeding either osteoblasts or osteocytes obtained by differentiating mesenchymal stem cells into the other half of the preliminary carrier; and

(4) seeding chondrocytes obtained by differentiating mesenchymal stem cells into one half of the preliminary carrier and subsequently seeding a cell suspension containing mesenchymal stem cells into the other half of the preliminary carrier and culturing and differentiating the mesenchymal stem cells on the carrier into either osteoblasts or osteocytes.

The preparation order of the chondrocytes and the osteoblasts or osteocytes may be reversed in any of the methods (1) through (4).

Differentiation of the mesenchymal stem cells into the chondrocytes is generally more difficult than differentiation of the mesenchymal stem cells into the osteoblasts. It is accordingly preferable to effectuate differentiation of the mesenchymal stem cells into the chondrocytes prior to differentiation into the osteoblasts. For the enhanced therapeutic response, it is essential that the implant material has a matrix equivalent to the matrix (extracellular matrix) produced in each implantation site. By taking into account this factor, in the case of seeding the chondrocytes obtained by differentiating the mesenchymal stem cells onto the carrier, the process requires further culture of the seeded chondrocytes for production of the matrix. The cartilage tissue at the joint has the hyaline cartilage trait. Three-dimensional culture of the chondrocytes seeded on the preliminary carrier is required to give the hyaline cartilage trait to the seeded chondrocytes. In such cases, the additional culture process of inducing the chondrocytes to produce the matrix and to have the hyaline cartilage trait is required, in addition to the standard culture process of differentiating the mesenchymal stem cells into chondrocytes (two-stage culture). In the case of seeding a cell suspension containing the mesenchymal stem cells onto the preliminary carrier and culturing the seeded mesenchymal stem cells on the preliminary carrier, on the other hand, the culture simultaneously proliferates the mesenchymal stem cells and differentiates the mesenchymal stem cells into chondrocytes having the hyaline cartilage trait and producing the matrix. This requires only one-stage culture. This is why the latter procedure is preferable. Namely the methods (1) and (2) are preferred to the methods (3) and (4).

The invention is also directed to a method of producing an implant material, which includes a cell-holding carrier that is formed in a three-dimensional shape and holds a cell thereon; a support member that is provided to surround the cell-holding carrier and supports the cell-holding carrier in an externally accessible state; and an artificial graft in a three-dimensional shape that is arranged adjacent to the cell-holding carrier, where the artificial graft is artificial bone and the cell is chondrocyte. This implant material production method makes the artificial bone constructed of an artificial bone material and subsequently prepares the cell-holding carrier with the chondrocyte held thereon to be arranged adjacent to the artificial bone and surrounded by the support member. It is desirable that an adhesive factor, such as fibronectin, is interposed between the artificial bone and the cell-holding carrier.

### BRIEF DESCRIPTION OF THE DRAWINGS

FIG. 1 is a perspective view schematically illustrating a base material for tissue regeneration in Example 1;

FIG. 2 is a perspective view schematically illustrating an implant material (bone/cartilage column) in Example 2;

FIG. 3 shows a general process of producing the implant material (bone/cartilage column) in Example 2;

FIG. 4 is a perspective view schematically illustrating an implant material (bone/cartilage column) in Example 3;

FIG. 5 shows a general process of producing the implant material (bone/cartilage column) in Example 3;

### EXAMPLES

### Example 1

### Base Material for Tissue Regeneration

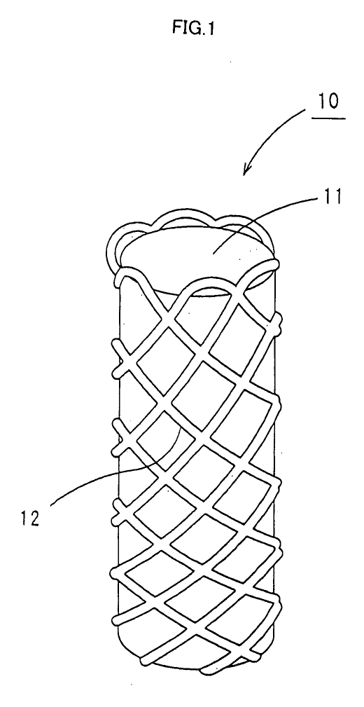

FIG. 1 is a perspective view schematically illustrating a base material for tissue regeneration. The base material for tissue regeneration 10 includes a collagen sponge 11 formed in a columnar shape and a mesh support member 12 provided to surround the periphery of the collagen sponge 11. The mesh support member 12 is composed of PLGA (polylactic acid-polyglycolic acid copolymer) and supports the collagen sponge 11 in an externally accessible state via its web structure. The base material for tissue regeneration 10 has an outer diameter of 6 mm and a height of 15 mm.

### Example 2

### Implant Material 1

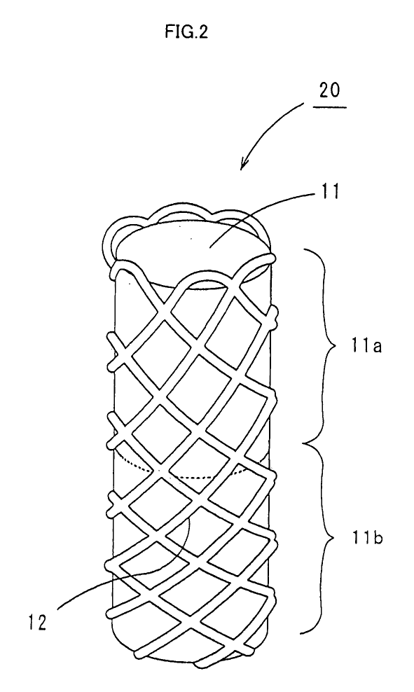

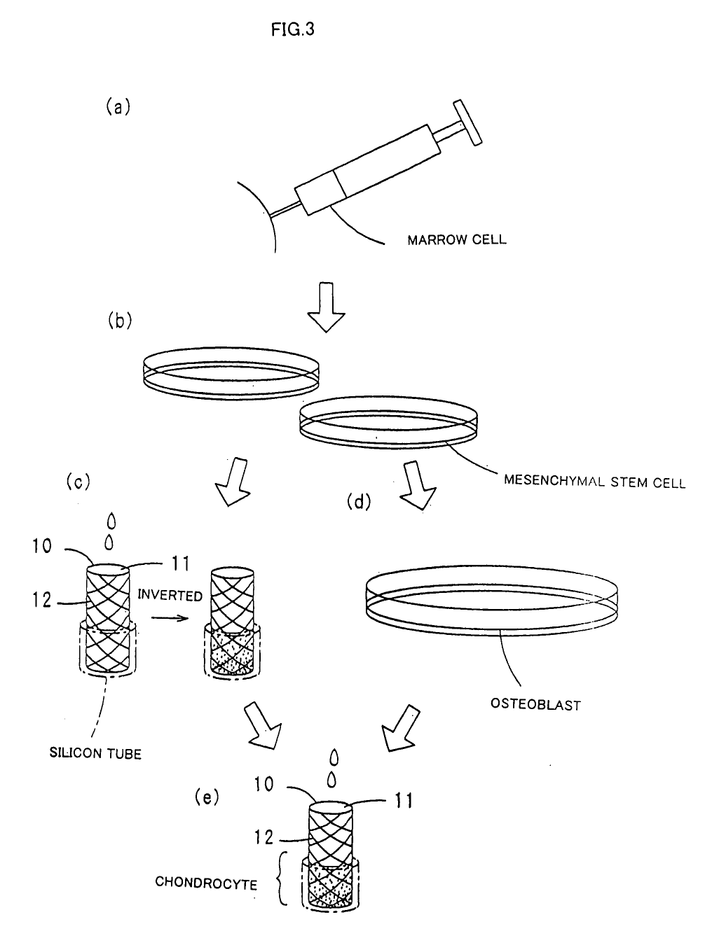

FIG. 2 is a perspective view schematically illustrating an implant material of one example. FIG. 3 shows a general process of producing the implant material of the example. This example produced an implant material (hereafter referred to as bone/cartilage column) 20 having the base material for tissue regeneration 10 of Example 1 as a scaffold of cell proliferation, chondrocytes held in a half area 11a of its collagen sponge 11, and osteoblasts held in the other half area 11b, as shown in FIG. 2. The process of preparing the bone/cartilage column 20 is described below with reference to FIG. 3.

The process first sucked marrow cells from the tibia of Japanese white rabbits with a syringe containing a small amount of heparin (see FIG. 3(a)), and then diluted the sucked marrow cells to 10 times with a DMEM (Dulbecco's Modified Eagle Medium) containing 10% FBS (fetal bovine serum) to prepare a cell suspension. Antibiotics were added to the DMEM. The process seeded 10 ml of the cell suspension in each 10 cm-dish and cultured the cells in an atmosphere of 5% CO₂ at 37° C. for 1 week. After the 1-week culture, the culture medium was replaced. The marrow cells included blood cells and mesenchymal stem cells. The replacement of the culture medium removed only suspension blood cells and adhesion-dependent mesenchymal stem cells adhering to the bottom of the dish were isolated. After the first replacement of the culture medium, the culture medium was replaced at intervals of every 3 to 4 days for proliferation of the mesenchymal stem cells (see FIG. 3(b)). In order to obtain a sufficient amount of cells, subculture was carried out according to the requirements. The subculture gives a sufficient amount of mesenchymal stem cells. Proliferation of the mesenchymal stem cells over the whole culture surface of the dish (to the state of confluent growth) starts differentiation of the stem cells. The proliferation was repeated until the state of sufficient but not confluent growth and the mesenchymal stem cells were kept in the undifferentiated state.

After proliferation of a sufficient amount of mesenchymal stem cells, the mesenchymal stem cells were detached from the bottom of the dish by trypsin treatment for 5 minutes. The detached mesenchymal stem cells were subjected to centrifugation at 1500 rpm for 5 minutes. The obtained pellets of the mesenchymal stem cells were suspended in a chondrocyte differentiation-inducing medium to prepare a cell suspension having the cell density of not less than 4×10⁷ cells/ml. The chondrocyte differentiation-inducing medium used here was a serum-free medium obtained by adding 10⁻⁸ M dexamethasone, 10⁻⁵ M β-glycerophosphate, 0.05 mg/ml ascorbic acid-2-phosphate, and antibiotics to a DMEM.

The base material for tissue regeneration 10 described above was set in each sterile silicon tube (outer diameter: 10 mm, inner diameter: 6 mm, height: 7 mm) placed on a 24-well plate. Namely the silicon tube supported the base material for tissue regeneration 10 upright (see FIG. 3(c)). The process then added dropwise 100 μl of the cell suspension (mesenchymal stem cells+chondrocyte differentiation-inducing medium) prepared as discussed above onto the collagen sponge 11 of the base material for tissue regeneration 10 with an Eppendorf pipette. The dropping of the volume 100 μl caused the cell suspension to permeate into only an upper half of the collagen sponge 11 and made the mesenchymal stem cells adhere to the upper half of the collagen sponge 11, while keeping a lower half of the collagen sponge 11 free from the mesenchymal stem cells. The base material for tissue regeneration 10 in this state was inverted to face down the cell seeding area. The inverted base material for tissue regeneration 10 was supported upright by the silicon tube in the same manner as before. With elapse of 1 hour since the inversion, the mesenchymal stem cells were fixed to the base material for tissue regeneration 10. After adding of 1.5 ml of the chondrocyte differentiation-inducing medium having the above composition, the base material for tissue regeneration 10 with the mesencymal stem cells fixed thereon was cultured in an atmosphere of 5% CO₂ at 37° C. for 2 weeks. The culture medium was replaced every other day. On completion of the culture, the process fixed the base material for tissue regeneration 10 with formalin, made a paraffin-embedded tissue slice, and stained the tissue slice by the technique of alcian blue staining used for staining the cartilage matrix. Observation of the stained tissue slice showed the presence of stained acidic mucopolysaccharides specifically produced by the chondrocytes. This proved adequate differentiation of the mesenchymal stem cells into the chondrocytes.

In the meanwhile, the process cultured the mesenchymal stem cells in some of the 10 cm-dishes to the state of confluent growth. After the confluent growth of the mesenchymal stem cells, the culture medium for cell proliferation was replaced with an osteoblast differentiation medium. The culture in an atmosphere of 5% CO₂ at 37° C. for 2 weeks differentiated the mesenchymal stem cells into osteoblasts (see FIG. 3(d)). The osteoblast differentiation medium used here was prepared by adding 10⁻7 M dexamethasone, 0.15 mM ascorbic acid 2-phosphate, 1 mM pyruvic acid, 10 ng/ml rh TGF-β1 (recombinant human TGF-β), and 1/100 vol. (corresponding to 1/100 of the medium volume) of ITS premix (manufactured by Nippon Becton Dickinson Company, Ltd.) to a DMEM containing 10% FBS.

The differentiated osteoblasts were detached from the bottom of the dish by trypsin and collagenase treatment, and were mixed with antibiotics and a 10% FBS/DMEM to prepare an osteoblast suspension having the cell density of 2×10⁷ cells/ml. The osteoblast suspension thus obtained was added dropwise onto the base material for tissue regeneration 10 with the chondrocytes fixed in the half area of its collagen sponge 11. At this moment, the osteoblast suspension was dropped into the opposite half of the collagen sponge 11 where the chondrocytes held (see FIG. 3(e)). The base material for tissue regeneration 10 was stood still for 1 hour after the dropping of the osteoblast suspension. The osteoblasts were thus fixed on the collagen sponge 11. This gave the bone/cartilage column 20 having the chondrocytes held in the half area 11a of the collagen sponge 11 and the osteoblasts held in the other half area 11b as shown in FIG. 2.

After seeding of the osteoblasts, the process cultured the seeded osteoblasts in a DMEM containing 10% FBS with antibiotics added thereto to induce production of the matrix from the osteoblasts. This gave the bone/cartilage column 20 having the chondrocytes held in the half area 11a of the collagen sponge 11 and the osteocytes held in the other half area llb.

### Example 3

### Implant Material 2

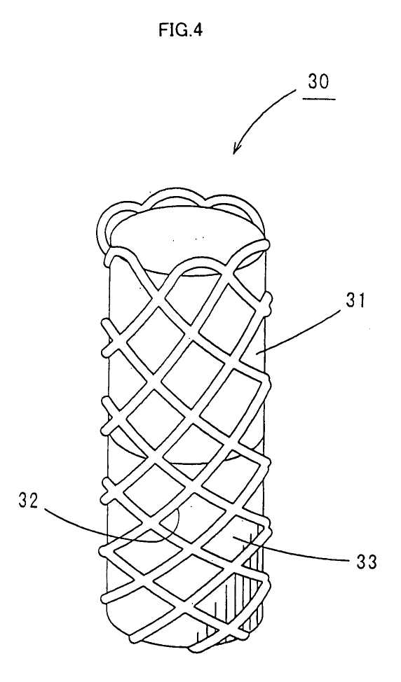

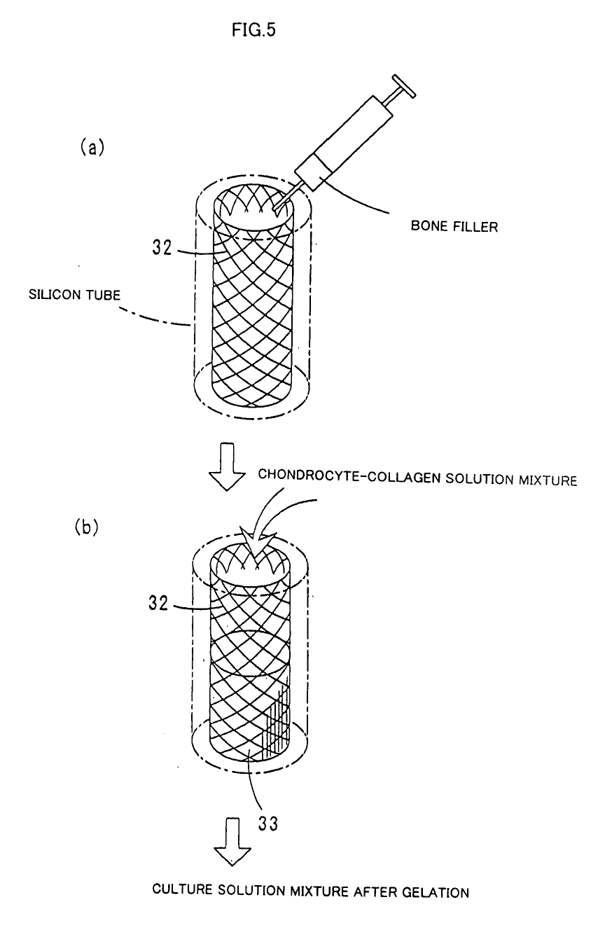

FIG. 4 is a perspective view schematically illustrating an implant material of another example. FIG. 5 shows a general process of producing the implant material of the example. This example produced a bone/cartilage column 30 as an implant material having a collagen gel 31 with chondrocytes held therein, an artificial bone 33 located below the collagen gel 31, and a mesh support member 32 formed to surround the collagen gel 31 and the artificial bone 33, as shown in FIG. 4. The process of preparing the bone/cartilage column 30 is described below with reference to FIG. 5.

The mesh support member 32 composed of PLGA (polylactic acid-polyglycolic acid copolymer) was set in each sterile silicon tube (outer diameter: 10 mm, inner diameter: 6 mm, height: 15 mm) placed on a 24-well plate. Namely the silicon tube supported the mesh support members 32 upright (see FIG. 5(a)).

The process then filled the mesh support member 32 surrounded by the silicon tube with 200 μl of a bone filler paste (trade name: Biopex manufactured by Mitsubishi Materials Corp.) containing calcium phosphate as its major component. The height of the bone filler approximated to 7 mm. The mesh support member 32 filled with the bone filler was stood still for about 10 minutes. The bone filler was then hardened to form hydroxyapatite, that is, the artificial bone 33 (see FIG. 5(b)).

In the meanwhile, the process obtained cartilage tissues from the joints of Japanese white rabbits, enzyme-treated the obtained cartilage tissues with a trypsin EDTA solution and a collagenase solution, and isolated chondrocytes. The isolated chondrocytes were washed and were mixed with a DMEM containing 10% FBS to prepare a cell suspension having the cell density of 4×10⁶ cells/ml. The process mixed the cell suspension thus obtained and 3% atelocollagen implant (manufactured by KOKEN Co., Ltd.) at a mixing rate of 1 to 1 to prepare a chondrocyte-collagen solution mixture. This mixing step diluted the cell density from 4×10⁶ cells/ml to 2×10⁶ cells/ml. The process filled 200 μl of the chondrocyte-collagen solution mixture onto the artificial bone 33 held in the mesh support member 32 described above. An adhesive factor, such as fibronectin, was placed on the artificial bone 33 in the process of filling the chondrocyte-collagen solution mixture.

The chondrocyte-collagen solution mixture on the artificial bone 33 was stood still in an atmosphere of 5% CO₂ at 37° C. for 1 hour to gelate. The gel was added with a medium and was cultured for 3 weeks to induce the chondrocytes to produce the matrix. This gave a bone/cartilage column. The medium used here was a 10% FBS-DMEM containing 50 μg/ml ascorbic acid.

### Example 4

### Evaluations of Bone/Cartilage Column

The bone/cartilage column (having the cartilage tissue in one half and the bone tissue in the other half) prepared in Example 2 was actually implanted in bone/cartilage defects of rabbits for evaluation of recovery. The evaluation test procedure anaesthetized each Japanese white rabbit (27 weeks old), incised the knee joint to disjoint patella, exposed the femur, and drilled the center of patella groove to make a full-thickness defect of 5 mm in diameter and 8 mm in depth. The bone/cartilage column 20 of Example 2 was cut to the depth of the defect and was implanted in the bone/cartilage defect. The mesh of the PLGA mesh support member 12 was stitched with and fixed to the surrounding healthy cartilage tissue with a PLGA suture thread. As a control group, the base material for tissue regeneration 10 without seeding of the cells was implanted in each bone/cartilage defect and was stitched with and fixed to the surrounding healthy cartilage tissue. Each surgery site was washed with physiological saline containing antibiotics and the cut was then stitched up. Both the bone/cartilage columns and the base materials for tissue regeneration had sufficient strength and were readily handled without deformation.

After 84 days in captivity, the rabbits were sacrificed under anesthesia. The procedure cut off the knee joint of the femur including the implantation site, made a tissue specimen, and fixed the tissue specimen with formalin. The procedure then stained each tissue slice by the technique of alcian blue staining and safranin 0 staining and made the histological observation. In the bone/cartilage column implantation group, the defects were positively stained with alcian blue and safranin 0. This revealed the recovery of the bone tissue and the cartilage tissue by means of the bone/cartilage column. In the control group, however, no sufficient recovery was observed. The recovery of the tissue by the growth of cells from the surrounding healthy tissue may be expected in the control group after the longer captivity.

Following the above procedure, the bone/cartilage column 30 (having the cartilage tissue in one half and the artificial bone in the other half) prepared in Example 3 was actually implanted in bone/cartilage defects of rabbits for evaluation of recovery. The evaluation test revealed the recovery of the defects in the implantation sites of the rabbits with the bone/cartilage column after 84 days.

The above examples are to be considered in all aspects as illustrative and not restrictive. There may be many modifications, changes, and alterations without departing from the scope or sprit of the main characteristics of the present invention. All changes within the meaning and range of equivalency of the claims are therefore intended to be embraced therein.

1. A base material for tissue regeneration, comprising:
a porous carrier that is formed in a three-dimensional shape; and
a support member that is provided to surround said porous carrier and supports said porous carrier in an externally accessible state.

2. A base material for tissue regeneration in accordance with claim 1, wherein said support member is any of a mesh support member, a palisade support member, and a perforated plate support member.

3. A base material for tissue regeneration in accordance with claim 1, wherein at least one of said porous carrier and said support member is composed of either of a biocompatible material and a bioabsorbable material.

4. A base material for tissue regeneration in accordance with claim 1, wherein said porous carrier is made of one component or a combination of multiple components selected from the group consisting of collagen, collagen derivatives, hyaluronic acid, hyaluronates, chitosan, chitosan derivatives, polyrotaxane, polyrotaxane derivatives, chitin, chitin derivatives, gelatin, fibronectin, heparin, laminin, and calcium alginate, and said support member is made of one component or a combination of multiple components selected from the group consisting of polylactic acid, polyglycolic acid, polycaprolactone, polylactic acid-polyglycolic acid copolymer, polylactic acid-polycaprolactone copolymer, and polyglycolic acid-polycaprolactone copolymer.

5. A base material for tissue regeneration in accordance with claim 1, wherein said support member has at least one suture thread.

6. A base material for tissue regeneration in accordance with claim 5, wherein the suture thread is composed of either of a biocompatible material and a bioabsorbable material.

7. A base material for tissue regeneration in accordance with claim 1, said base material being formed in a specific shape available for arthroscopic surgery.

8. An implant material, comprising:
a cell-holding carrier that is formed in a three-dimensional shape and holds a cell thereon; and
a support member that is provided to surround said cell-holding carrier and supports said cell-holding carrier in an externally accessible state.

9. An implant material in accordance with claim 8, wherein said cell-holding carrier is a porous carrier in a three-dimensional shape with the cell held thereon.

10. An implant material in accordance with claim 8, said implant material further comprising:
an artificial graft in a three-dimensional shape that is arranged adjacent to said cell-holding carrier.

11. An implant material in accordance with claim 8, wherein the cell includes at least one of chondrocyte, osteoblast, osteocyte, their precursor cells, mesenchymal stem cell, and embryonic stem cell (ES cell).

12. An implant material in accordance with claim 8, wherein the cell includes chondrocyte held in one half of said cell-holding carrier and either of osteoblast and osteocyte held in the other half of said cell-holding carrier.

13. An implant material in accordance with claim 10, wherein said artificial graft is artificial bone, and the cell is chondrocyte.

14. An implant material in accordance with claim 12, said implant material being applied to treatment of a bone/cartilage defect at a joint.

15. An implant material in accordance with claim 13, said implant material being applied to treatment of a bone/cartilage defect at a joint.

16. An implant material in accordance with claim 8, wherein said support member is any of a mesh support member, a palisade support member, and a perforated plate support member.

17. A base material for tissue regeneration in accordance with claim 8, wherein at least one of said carrier and said support member is composed of either of a biocompatible material and a bioabsorbable material.

18. An implant material in accordance with claim 8, wherein said carrier is made of one component or a combination of multiple components selected from the group consisting of collagen, collagen derivatives, hyaluronic acid, hyaluronates, chitosan, chitosan derivatives, polyrotaxane, polyrotaxane derivatives, chitin, chitin derivatives, gelatin, fibronectin, heparin, laminin, and calcium alginate, and said support member is made of one component or a combination of multiple components selected from the group consisting of polylactic acid, polyglycolic acid, polycaprolactone, and polylactic acid-polyglycolic acid copolymer.

19. An implant material in accordance with claim 8, wherein said support member has at least one suture thread.

20. An implant material in accordance with claim 19, wherein the suture thread is composed of either of a biocompatible material and a bioabsorbable material.

21. An implant material in accordance with claim 8, said implant material being formed in a specific shape available for arthroscopic surgery.

22. An implant material production method that produces an implant material comprising a cell-holding carrier that is formed in a three-dimensional shape and holds a cell thereon; and a support member that is provided to surround said cell-holding carrier and supports said cell-holding carrier in an externally accessible state,
said implant material production method adopting the process of:
differentiating mesenchymal stem cell into an object cell, preparing a cell suspension of the differentiated cell, and seeding the prepared cell suspension onto a preliminary carrier, which is capable of holding a cell and is formed in a three-dimensional shape, so as to obtain said cell-holding carrier.

23. An implant material production method that produces an implant material comprising a cell-holding carrier that is formed in a three-dimensional shape and holds a cell thereon; and a support member that is provided to surround said cell-holding carrier and supports said cell-holding carrier in an externally accessible state,
said implant material production method adopting the process of:
seeding a cell suspension containing mesenchymal stem cell onto a preliminary carrier, which is capable of holding a cell and is formed in a three-dimensional shape, and differentiating the mesenchymal stem cell held in said preliminary carrier into an object cell, so as to obtain said cell-holding carrier.

24. An implant material production method in accordance with claim 23, wherein said preliminary carrier is said porous carrier included in a base material for tissue regeneration comprising said porous carrier that is formed in a three-dimensional shape; and a support member that is provided to surround said porous carrier and supports said porous carrier in an externally accessible state.

25. An implant material production method that produces an implant material comprising a cell-holding carrier that is formed in a three-dimensional shape and holds a cell thereon; and a support member that is provided to surround said cell-holding carrier and supports said cell-holding carrier in an externally accessible state, wherein the cell includes chondrocyte held in one half of said cell-holding carrier and either of osteoblast and osteocyte held in the other half of said cell-holding carrier,
said implant material production method adopting the process of:
seeding a cell suspension containing mesenchymal stem cell into one half of a preliminary carrier, which is capable of holding a cell and is formed in a three-dimensional shape, and culturing and differentiating the mesenchymal stem cell on said preliminary carrier to make chondrocyte held in the one half of said preliminary carrier, and subsequently seeding either of osteoblast and osteocyte differentiated from the mesenchymal stem cell into the other half of said preliminary carrier, so as to obtain said cell-holding carrier.

26. An implant material production method that produces an implant material comprising a cell-holding carrier that is formed in a three-dimensional shape and holds a cell thereon; and a support member that is provided to surround said cell-holding carrier and supports said cell-holding carrier in an externally accessible state, wherein the cell includes chondrocyte held in one half of said cell-holding carrier and either of osteoblast and osteocyte held in the other half of said cell-holding carrier,
said implant material production method adopting the process of:
seeding a cell suspension containing mesenchymal stem cell into one half of a preliminary carrier, which is capable of holding a cell and is formed in a three-dimensional shape, and culturing and differentiating the mesenchymal stem cell on said preliminary carrier to make chondrocyte held in the one half of said preliminary carrier, and subsequently seeding a cell suspension containing mesenchymal stem cell into the other half of said preliminary carrier and culturing and differentiating the mesenchymal stem cell on said preliminary carrier into either of osteoblast and osteocyte, so as to obtain said cell-holding carrier.

27. An implant material production method in accordance with claim 26, wherein said preliminary carrier is said porous carrier included in a base material for tissue regeneration comprising said porous carrier that is formed in a three-dimensional shape; and a support member that is provided to surround said porous carrier and supports said porous carrier in an externally accessible state.

28. An implant material production method that produces an implant material comprising a cell-holding carrier that is formed in a three-dimensional shape and holds chondrocyte thereon; a support member that is provided to surround said cell-holding carrier and supports said cell-holding carrier in an externally accessible state; and an artificial bone in a three-dimensional shape that is arranged adjacent to said cell-holding carrier,
said implant material production method making the artificial bone constructed of an artificial bone material and subsequently preparing said cell-holding carrier with the chondrocyte held thereon to be arranged adjacent to the artificial bone and surrounded by said support member.
